(19) United States
(12) Patent Application Publication
(10) Pub. No.: US 2003/0236527 A1
(43) Pub. Date: Dec. 25, 2003

(54) Multi-slot guide for bone-setting operation for a femoral neck fracture

(76) Inventor: Fujio Kawakami, Ube-shi (JP)

Correspondence Address: KUBOVCIK & KUBOVCIK, SUITE 710, 900 17TH STREET NW, WASHINGTON, DC, 20006

(21) Appl. No.: 10/176,545

(22) Filed: Jun. 24, 2002

(51) Int. Cl. A61B 17/58

(52) U.S. Cl. 606/96

(57) ABSTRACT

An object of the present invention is to provide a multi-slot guide for a femoral neck fracture bone-setting operation, by which a high level of fixation of, for example, a guide pin to a femoral head part can be obtained with no regard to the size of the femur.

During a femoral neck fracture bone-setting operation, in the multi-slot guide 10 for the femoral neck fracture bone-setting operation, pin inserting slots a1, d1 and g1 are selected so as to form a triangle under the condition that each of them may be positioned in the site corresponding to the lateral cortical bone S. Then, the guide pins 13A to 13C are inserted from the base region of the femoral greater trochanter G through the locations having contacts with said lateral cortical bone S into the inner region of the femoral head H via respective pin inserting slots a1, d1 and g1. Consequently, the guide pins 13A to 13C and thus the screws 16A to 16C can be rigidly fixed to the upper region of the femur in match with the size of individual femur. Therefore, earlier application of the load to the affected site becomes possible.

### FIELD OF THE INVENTION

The present invention relates to a multi-slot guide for bone-setting operation for a femoral neck fracture, and specifically to a multi-slot guide used in the cannulated cancellous hip screw technique.

### DESCRIPTION OF THE PRIOR ART

In the operations taken on the femoral neck region for the fracture, in which serious displacement are often observed for the aged persons, a technique of replacing a head part of femur (hereafter, sometimes referred to as femoral head) separated from an upper region of the femur due to the fracture with an artificial femoral head has been frequently used, taking a dim view of the risk of false joint or femoral head necrosis.

However, this technique has the following problems. That is, in this technique, bone marrow within the femur is seriously invaded, leading to a poor blood circulation within the bone marrow. Further, since this artificial femoral head has a limited lifetime, and therefore another operation will be necessary to change the artificial femoral head after a certain period has elapsed. This may cause another problem, rising of medical expenses.

In recent years, the femoral bone-setting technique for preserving the femoral head as remained in the site has been getting known as one surgical technique aiming for resolving those problems. The cannulated cancellous hip screw technique, which may be sometimes abbreviated to CCHS technique, has been developed as one of this type of techniques.

The CCHS technique uses a multi-slot guide for the femoral neck fracture bone-setting operation (hereafter, sometimes referred to as simply a multi-slot guide), in which three screws arranged in parallel-relationship to form a triangle are screwed into the upper region of the femur, thereby securing the separated femoral head onto the upper region of the femur.

Referring to FIG. 11, a prior art multi-slot guide 100 will be described. FIG. 11 is a front view of a multi-slot guide for the femoral neck fracture bone-setting operation according to the prior art.

As shown in FIG. 11, the prior art multi-slot guide 100 has a guide main body 101 made of metal having a short length and a circular shape in sectional view, and a hand holder 101a for holding the multi-slot guide 101. In the guide main body 101, total of six-pin inserting slots x1, x2, x3, x4, x5 and x6 are formed by every 60 degrees around a guide axis line penetrating through the peripheral portions in either end surface of the main body 101. It is to be noted that respective pin inserting slots x1˜x6 are the slots of ø3.0 mm formed in parallel relationship to one another. In FIG. 11, x7 designates a seventh pin inserting slot disposed so as to penetrate though the guide main body 101 along the axial line. This pin inserting slot x7 is arranged for accepting a guide pin, so that the guide pin may penetrate through the femoral neck region and the femoral head in their central locations for the purpose of securely holding the fracture site as well as for rotating the multi-slot guide 100 around that guide pin as the center.

A specific technical procedure of the CCHS will be described below with reference to FIG. 11. All of the procedures of this operation may be carried out while checking the radioscopic x-ray image on a monitor screen.

Primarily, a long guide pin having a diameter of ø3.0 mm is inserted from a base region of femoral greater trochanter (sometimes referred to as greater trochanter) through the femoral neck region into the femoral head. This insertion may be performed in the form of hammering-in with a hammer.

Subsequently, the prior art multi-slot guide 100 is introduced along the first guide pin such that the first guide pin may be inserted through the pin inserting slot x3. Through this step, the multi-slot guide 100 is fitted onto the first guide pin.

In the condition with the first guide pin being inserted in the pin inserting slot x3, an insertion point for a second guide pin is selected so that all of the three guide pins can be inserted into the femoral neck region, while rotating the multi-slot guide 100 in either direction with the first guide pin positioned in the center.

It is to be appreciated that once the insertion point of the second pin has been selected, in normal cases, the insertion point for a third guide pin could be also determined. Typically, for the insertion points of the second and the third guide pins, the pin inserting slot x1 (for the second pin insertion) and the pin inserting slot x5 (for the third pin insertion) would be selected respectively, which are located on the left side and the right side of the pin inserting slot x3 so as to form an angle of 60 degrees therebetween around the slot x3.

After this step, a guide pin of ø3.0 mm is inserted in the pin inserting slot x1 for the second pin toward the femoral neck region and hammered thereinto. Then, the third guide pin is inserted in the pin inserting slot x5 for the third pin and then hammered into the upper region of the femur.

Subsequently, the multi-slot guide 100 is taken away from the guide pins, and with using those three guide pins as the guiding axes, three of tubular screws are screwed into the upper region of the femur, while still keeping the triangle and the parallel relationship to one another. In a sequence of the screwing step, the tubular screw mounted on the first guide pin may be screwed in first, followed by the screw mounted on the second guide pin and then the screw mounted on the third guide pin. It is to be appreciated that the length of the screw to be used is determined by taking into consideration, for example, a run-through length of a Kirschner's wire drawn from the upper region of the femur.

Thus, the femoral head once separated from the upper region of the femur has been rigidly secured to this upper region of the femur by means of the screws.

It is to be appreciated that the size of the femur subject to such CCHS surgical technique is, as the matter of course, different individually, depending on the age, sex, height and body build of each individual patient. Further, the type of fracture is diversely differentiated, and some are of highly unstable type. Preferably, the operation should be performed such that the maximum fixation may be obtained corresponding to each case.

However, according to this multi-slot guide 100, the respective six pin inserting slots x1˜x6 are disposed in the periphery of the guide main body 101 along the same imaginary circle centered on the axis line of the multi-slot guide 100. The area of the imaginary equilateral triangle formed by the three guide pins is regularly constant. Accordingly, it is unable to handle with each differently sized femur of the patient, and therefore a standardized operation for inserting the Kirschner's wires and thus the guide pins and screws have been practically conducted.

Inconsequence, the screw may pass through the center of the femoral neck region having lower bone density, and thereby there have been such problems that unsatisfactory fixation occurs in many cases and that the femoral head bone marrow is invaded seriously (see FIG. 12 illustrating the location of the three guide pins inserted into the femoral neck region according to the prior means).

Besides, a principal object of this prior art multi-slot guide 100 is to guide the three guide pins so that they may be inserted into respective positions in the bone marrow of the femoral neck region so as to form the equilateral triangular therein while keeping the parallel relationship to one another as much as possible.

In the light of the aforementioned circumstances, an object of the present invention is to provide a multi-slot guide for the femoral neck fracture bone-setting operation, in which a high level of fixation of the guide pins and thus the screws to the femoral head can be obtained irrespective of the size of the individual femur.

Another object of the present invention is to provide a multi-slot guide for the femoral neck fracture bone-setting operation, in which upon inserting the third guide pins into the femur, the third guide pins can be regularly inserted in parallel relationship with respect to the other guide pins and also a high level of fixation of the guide pins and thus the screws can be obtained irrespective of the size of individual femur.

### SUMMARY OF THE INVENTION

According to a first aspect of the present invention, there is provided a multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins, which have been inserted from a base region of a femoral greater trochanter through the femoral neck region into the femoral head part, as guiding axes upon screwing-in, said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them, wherein said respective pin inserting slots are formed penetrating through said multi-slot guide so as to be disposed along extension lines connecting an imaginary center-axis line of said multi-slot guide for the femoral neck fracture bone-setting operation with respective vertexes of imaginary triangles formed around said imaginary center-axis line such that a plurality of pin inserting slots is formed along respective extension lines, said extension lines being arranged radially starting from said imaginary center-axis line at respective angle positions of 0 degree, 60 degree, 90 degree, 120 degree, 150 degree, 180 degree, 240 degree, 270 degree, 300 degree, and 330 degree.

Such properties of the multi-slot guide for the femoral neck fracture bone-setting operation as shape, material, size and so forth are not limited. For example, the multi-slot guide may comprise a guide main body of metal column having a circular section with a diameter in a range of 5 to 10 cm and a length in a range of 3 to 15 cm, in which a plurality of pin inserting slots are formed so as to penetrate through the guide main body along respective slot axes parallel with the axial line of the guide main body.

The number of pin inserting slots to be formed is not limited. For example, 10 or 20 of slots may be formed. Locations for respective pin inserting slots to be formed are arbitrarily determined so far as each of said pin inserting slots is located on one of said extension lines arranged radially starting from said imaginary center-axis line at respective angle positions of 0 degree, 60 degree, 90 degree, 120 degree, 150 degree, 180 degree, 240 degree, 270 degree, 300 degree, and 330 degree. Further, the penetrating directions of respective pin inserting slots are also not limited.

Still further, bore diameters of respective slots are not limited. Typically, they may be modified to fit to the diameters of the guide pins to be inserted.

It is to be appreciated that the imaginary center line is not necessarily identical with the axial line of this multi-slot guide for the femoral neck fracture bone-setting operation. This means that the imaginary center line may be located in an eccentric location of this multi-slot guide.

According to this invention, the pin inserting slots for the three guide pins, which should be arranged to form a triangle, can be selected during the bone-setting operation on the femur. At that time, the pin inserting slots for the three pins are properly selected so that respective pin inserting slots may match with corresponding sites of the lateral cortical bone in the femoral neck region.

After that step, the guide pins are introduced into respective selected pin inserting slots, and then respective guide pins are inserted from the base region of the femoral greater trochanter, passing though a location contacting with the inner edge of the lateral cortical bone of the femoral neck region, finally into the inside of the femoral head part.

Inserting the screws in contact with the inner edge of the lateral cortical bone allows the screws to obtain the support by the lateral cortical bone, so that the bone-setting site can be securely fixed. In specific, the locations for inserting the screws can be adjusted so as to match with the size of the femur of each individual patient, so that the screws can be inserted along the inner edge of the lateral cortical bone. Owing to this, an earlier application of the load to the affected site becomes possible and thus a period required until the patient is allowed to walk can be reduced.

It should be noted that the femoral neck fractures occur frequently in the aged people. According to the prior art technique, even if the surgical treatment by the CCHS technique has been curried out, a problematic condition still remains with the fixation and it must take a long time until the patient is allowed to start rehabilitation. Due to this condition, the aged people have developed coincidentally internal medical complications in high frequencies and/or the late functional recovery and the extended hospitalization period have been obstacles to returning the patients back to their independent livings. Besides, since the artificial femoral head replacement operation with deep invasion has been often chosen specifically in the femoral neck fracture of the unstable type associated with serious displacement, the complications have been more frequently developed during and/or after the operation, and in this point of view, there has still been a problem with the operational achievement in spite of increase in the medical expenses. The present invention can improve those problematic conditions.

According to this invention, since respective pin inserting slots are formed penetrating through said multi-slot guide body so as to be disposed along the extension lines connecting the imaginary center-axis line of the multi-slot guide with respective vertexes of the imaginary triangles formed around said imaginary center-axis line in such a manner that a plurality of pin inserting slots is formed along respective extension lines, a high level of fixation of the guide pins and thus the screws to the femoral head part can be obtained irrespective of the size of the individual femur. This enables the earlier application of the load to the site of interest and also the time period required until the patient is allowed to walk can be reduced.

According to a second aspect of the present invention, there is provided a multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins, which have been inserted from a base region of a femoral greater trochanter through the femoral neck region into the femoral head part, as guiding axes upon screwing-in, said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them, wherein said respective pin inserting slots comprise one reference slot and a plurality of differently distant slots each having an individual distance from said reference slot different from one another respectively.

According to a third aspect of the present invention, there is provided a multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins, which have been inserted from a base region of a femoral greater trochanter through the femoral neck region into the femoral head part, as guiding axes upon screwing-in, said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them, wherein said multi-slot guide for a femoral neck fracture bone-setting operation is of cylindrical shape with approximately equilateral triangular section and said respective pin inserting slots comprise one reference slot formed in a proximity of one of vertexes of said approximately equilateral rectangular and a plurality of differently distant slots each being arranged radially around said reference slot with an individual distance from said reference slot different from one another respectively.

The distances of respective differently distant slots defined as from the reference slot are not limited. For example, the distances of any adjacent slots from the reference slot may be differentiated by 1 mm, or by a few mm with respect to each other.

In an alternative embodiment according to the third aspect of the present invention, there is provided a multi-slot guide for a femoral neck fracture bone-setting operation, in which the distances of said differently distant slots defined from said reference slot increase by 2 mm in turn from the shortest one.

For example, these distances may be 12 mm, 14 mm, 16 mm, 18 mm, 20 mm, 22 mm, 24 mm, 26 mm, 28 mm, 30 mm and so on.

According to this invention, during the bone-setting operation of the femur, for example, after the first guide pin having been inserted along the lateral cortical bone in the femoral neck region, the multi-slot guide for the femoral neck fracture bone-setting operation is mounted on this first guide pin in such a manner that the first guide pin is inserted into the reference slot.

Then, with the first guide pin being inserted in the reference slot, a differently distant slot for a second guide pin corresponding to another location in the lateral cortical bone of this femur is selected. Subsequently, the second guide pin is introduced into this selected differently distant slot, and then inserted from the base region of the femoral greater trochanter so as to be brought into contact with said another location in the lateral cortical bone of the femur and finally into the femoral head part.

After that step, the selecting procedure of a differently distant slot for a third guide pin is performed. In specific, primarily, the multi-slot guide for the femoral neck fracture bone-setting operation is removed away from the first and the second guide pins. Then, the multi-slot guide is mounted again with the first guide pin being inserted into the reference slot. In this step, the second guide pin should not be inserted in any one of the differently distant slots.

After that, with the first guide pin remained in the reference slot, such a location for a third guide pin that maximizes an area of a triangle formed by the three guide pins must be found, and the differently distant slot satisfying this location should be selected as the inserting slot for the third guide pin. Then, the third guide pin is inserted through this differently distant slot so as to be brought into contact with the lateral cortical bone and finally into the femoral head part.

This allows the third guide pin to be inserted in parallel with the other guide pins in a smooth and reliable manner. In addition, the guide pins and thus the screws can be secured to the femoral head part irrespective of the size of individual femur. This allows the load to be applied to the site of interest in an earlier time. If the three guide pins are not inserted in the parallel relationship, an intensified power would not be applied for fixing the femoral head part which has been once separated from the upper region of the femur due to the fracture and the earlier load application may probably be problematic.

According to this invention, since respective pin inserting slots comprise one reference slot and a plurality of differently distant slots each having individual distance from the reference slot different from one another, respectively, therefore upon inserting the third guide pin into the femur, this third guide pin can be regularly inserted in parallel with the other guide pins, and further the high level of fixation of the guide pin and thus the screw with respect to the femoral head part can be accomplished irrespective of the size of individual femur. As a result, the earlier application of the load to the site of interest can be realized.

### BRIEF DESCRIPTION OF THE DRAWINGS

FIG. 1 is a schematic diagram of a multi-slot guide for a femoral neck fracture bone-setting operation according to a first embodiment of the present invention, illustrating a condition in practical use;

FIG. 2 is a perspective view of the multi-slot guide for the femoral neck fracture bone-setting operation according to the first embodiment of the present invention;

FIG. 3 is a schematic diagram illustrating an example of selection of three pin inserting slots with respect to the femur of standard size in the multi-slot guide for the femoral neck fracture bone-setting operation according to the first embodiment of the present invention;

FIG. 4 is a schematic diagram illustrating an example of selection of the three pin inserting slots with respect to the femur of large size in the multi-slot guide for the femoral neck fracture bone-setting operation according to the first embodiment of the present invention;

FIG. 5 is a longitudinal sectional view of the upper region of the femur, illustrating a condition where first guide pin is now being inserted by using the multi-slot guide for the femoral neck fracture bone-setting operation according to the first embodiment of the present invention;

FIG. 6 is a schematic diagram illustrating a location of the three guide pins inserted into the femoral neck region according to the first embodiment of the present invention;

FIG. 7 is a longitudinal sectional view of the upper region of the femur illustrating a condition where the Kirschner's wire for inserting a second guide pin is now being operated to pierce the femur by using the multi-slot guide for the femoral neck fracture bone-setting operation according to the first embodiment of the present invention;

FIG. 8 is longitudinal sectional view of the upper region of the femur illustrating a condition where the second guide pin is now being inserted by using the multi-slot guide for the femoral neck fracture bone-setting operation according to the first embodiment of the present invention;

FIG. 9 is a longitudinal sectional view of the upper region of the femur illustrating a condition where the screws have been screwed-in with utilizing as guiding axes the guide pins inserted by using the multi-slot guide for the femoral neck fracture bone-setting operation according to the first embodiment of the present invention;

FIG. 10(a) is a perspective view illustrating an insertion procedure of a second guide pin into the femur by using a multi-slot guide for a femoral neck fracture bone-setting operation according to a second embodiment of the present invention;

FIG. 10(b) is a perspective view illustrating the insertion procedure of a third guide pin into the femur by using the multi-slot guide for the femoral neck fracture bone-setting operation according to the second embodiment of the present invention;

FIG. 11 is a front view of a multi-slot guide for a femoral neck fracture bone-setting operation according to a prior art; and

FIG. 12 is a schematic diagram illustrating a location of the three guide pins inserted into the femoral neck region according to the prior art.

### DESCRIPTION OF THE PREFERRED EMBODIMENTS

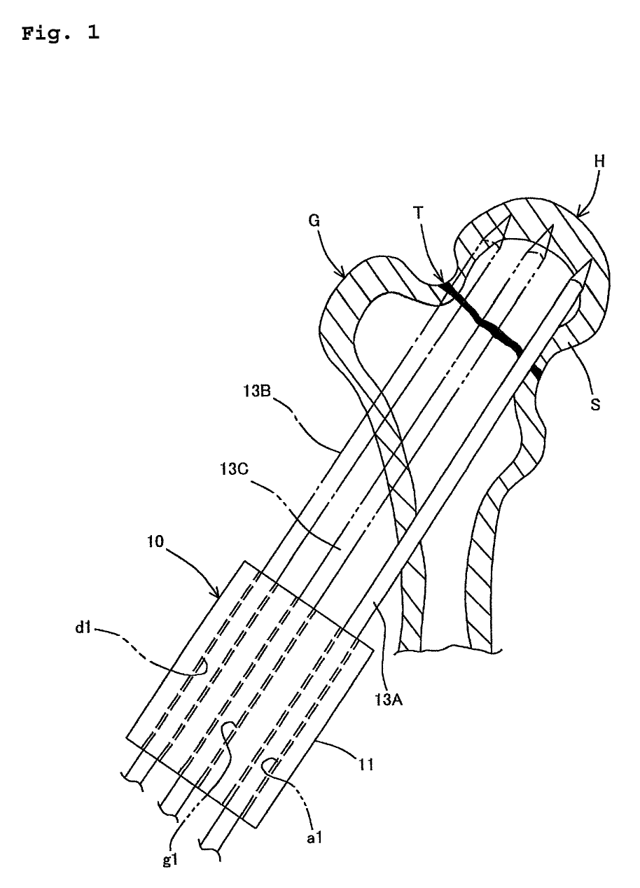

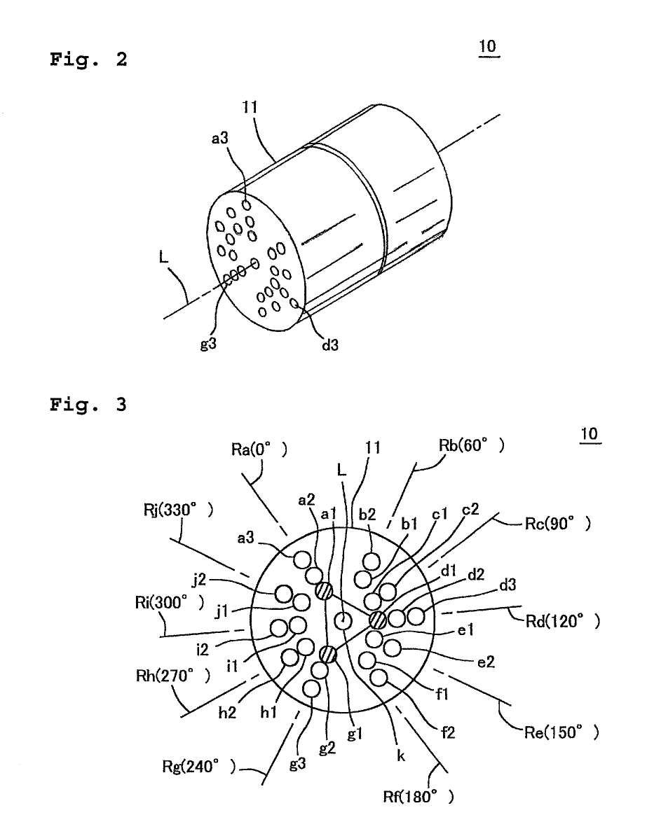

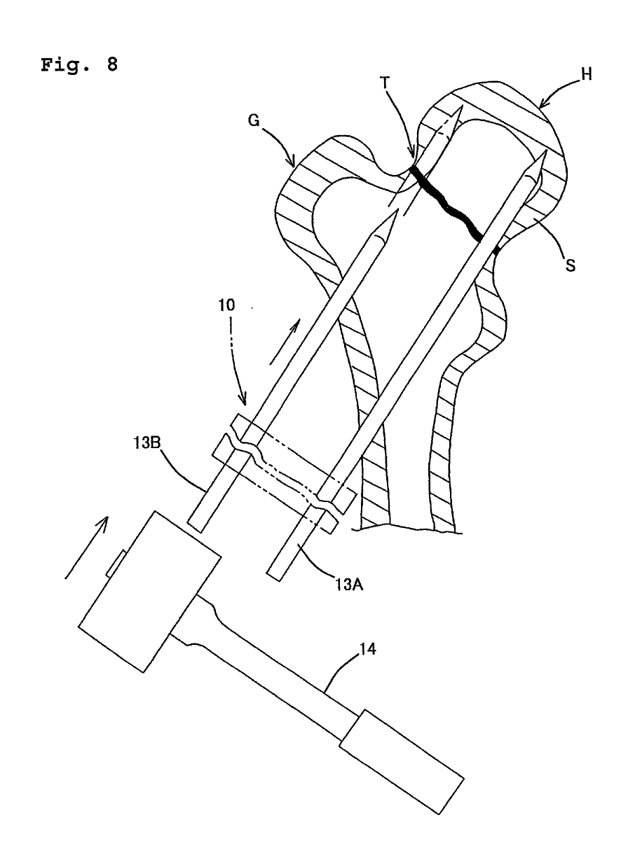

Preferred embodiments of the present invention will be described below with reference to the attached drawings. First of all, referring to FIGS. 1 to 9, a multi-slot guide for a femoral neck fracture bone-setting operation according to a first embodiment of the present invention will be described.

In FIGS. 1 to 3, reference numeral 10 designates a multi-slot guide for a femoral neck fracture bone-setting operation according to a first embodiment of the present invention (hereafter, sometimes referred to as a multi-slot guide), and this multi-slot guide 10 has a guide main body 11 in the form of short metal column having a circular section with a diameter of 4 cm and a length of 6 cm. In this guide main body 11, total of 23 pin inserting slots of ø3.0 mm, a1, a2, a3, b1, b2, c1, c2, d1, d2, d3, e1, e2, f1, f2, g1, g2, g3, h1, h2, i1, i2, j1 and j2, are formed respectively, so as to extend in parallel with one another and to penetrate through the main body 11 in peripheral portions in either end surface, wherein a plurality of inserting slots is disposed along respective extension lines connecting an imaginary center line “L” of the multi-slot guide 10 to respective vertexes of imaginary triangles formed around this imaginary center line L, respectively. It is to be noted that this imaginary center line L coincides with the axial line of the multi-slot guide 10. Along this imaginary center line L, a 24th pin inserting slot “k” is formed to penetrate through the guide main body 11. This pin inserting slot k is used to pass a guide pin therethrough so that the guide pin may penetrate the central regions of femoral neck and femoral head for the purpose of fixedly holding the fracture site as well as for rotating the multi-slot guide 10 around the guide pin (though typically not used). A specific arrangement of locations of those inserting slots a1˜j2 will now be described.

That is, in the first embodiment, there are 10 extension lines in total, i.e. the lines Ra, Rb, Rc, Rd, Re, Rf, Rg, Rh, Ri and Rj, extending in guide radial directions for interconnecting the imaginary center line L and the respective vertexes of the imaginary triangles. For example, assuming the line Ra is a reference line (defined at zero degree position), the line Rb is defined at a position having a 60-degree phase angle with respect to the reference line with the imaginary center line L as center, the line Rc at a position having a 90-degree phase angle, the line Rd at a position having a 120-degree phase angle, the line Re at a position having a 150-degree phase angle, the line Rf at a position having a 180-degree phase angle, the line Rg at a position having a 240-degree phase angle, the line Rh at a position having a 270-degree phase angle, the line Ri at a position having a 300-degree phase angle, and the line Rj at a position having a 330-degree phase angle, respectively.

On the lines Ra, Rd and Rg, there are three groups of pin inserting slot each including three slots respectively, the pin inserting slots a1, a2, a3, the pin inserting slots d1, d2 and d3 and the pin inserting slots g1, g2, g3, aligned along respective lines in serial with a 4 mm difference of distance from the imaginary center line L between adjacent two slots within the same group. Among those slots, the distance from the imaginary center line L to each of the pin inserting slots a1, d1 and g1 are all equal. The distance from the imaginary center line L to each of the pin inserting slots a2, d2 and g2 are also all equal. Further, the distance from the imaginary center line L to each of the pin inserting slots a3, d3 and g3 are also all equal.

On the other hand, along the other seven lines, the line Rb, the line Rc, the line Re, the line Rf, the line Rh, the line Ri and the line Rj, there are seven groups of inserting slot each including two slots, the inserting pin slots b1 and b2, the pin inserting slots c1 and c2, the pin inserting slots e1 and e2, the pin inserting slots f1 and f2, the pin inserting slots h1 and h2, the pin inserting slots i1 and i2, and the pin inserting slots j1 and j2, aligned along respective lines in serial. For example, on the radially extending line Rb, the pin inserting slot b1 is disposed on the radially inner side in alignment with the pin inserting slot b2 disposed radially on the outer side thereof.

The distances from those pin inserting slots b1, b2, c1, c2, e1, e2, f1, f2, h1, h2, i1, i2, j1 and j2 to the imaginary center line L (i.e., the radial distances) are varied respectively by small lengths.

FIG. 3 shows an example for selecting the three pin inserting slots with respect to the femur of standard size. In this case, the three guide pins may be inserted respectively, for example, into the pin inserting slots a1-d1-g1 forming a triangle having respective edge lengths of 14 mm.

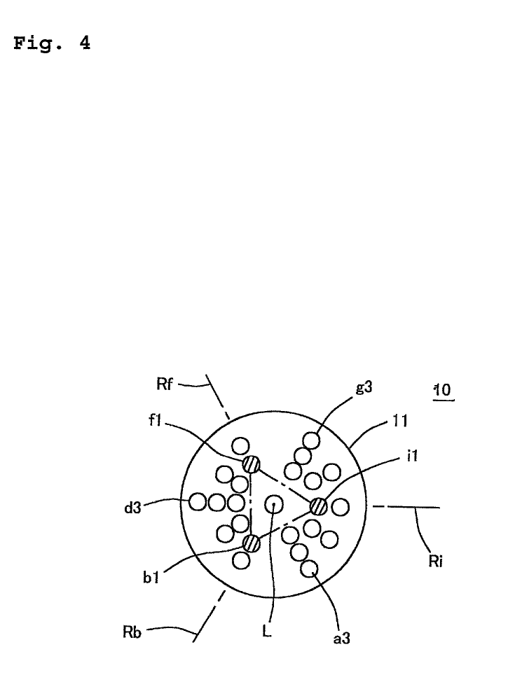

On the other hand, FIG. 4 shows an example for selecting the three pin inserting slots with respect to the femur of larger size. In FIG. 4, the three guide pins may be inserted respectively, for example, into the pin inserting slots b1-f1-i1 forming a triangle having respective edge lengths of 17.5 mm.

A bone-setting operation of the femur by way of the CCHS technique using the multi-slot guide 10 according to this first embodiment will now be described with reference to FIG. 1 and FIGS. 5 to 9. For the purpose of this illustration, the explanation is directed to the example for selecting the three pin inserting slots with respect to the femur of standard size of FIG. 3. This operation may be carried out while observing the site subject to the surgical treatment by the radioscopy.

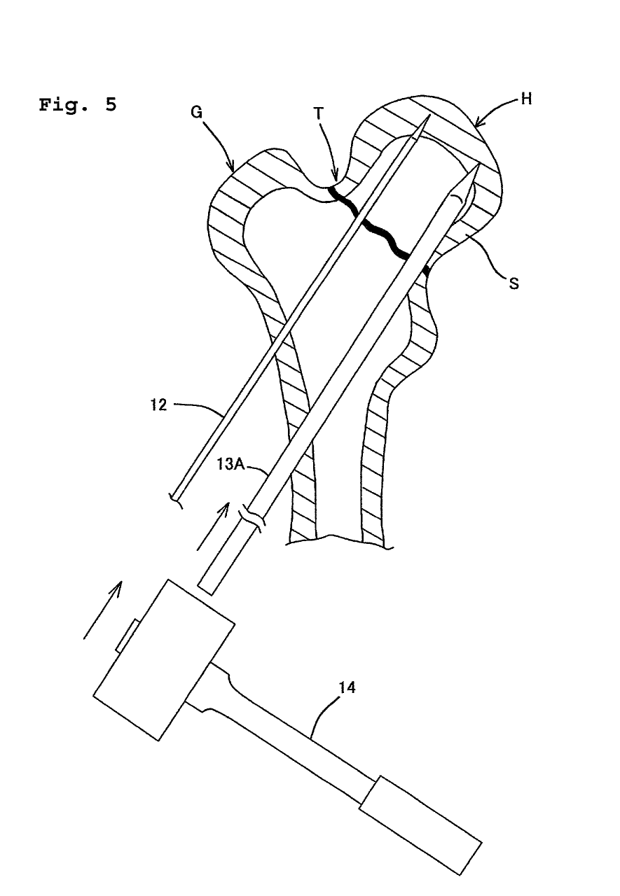

Primarily, as shown in FIG. 5, a thin Kirschner's wire 12 of ø1.5 mm is installed within soft tissue directly above the femoral neck region, and an optimal angle for inserting a guide pin into the femur is simulated by using the radioscopy. For example, the optimal angle may be in a range of 130 to 140 degrees with respect to the bone axis of the femur.

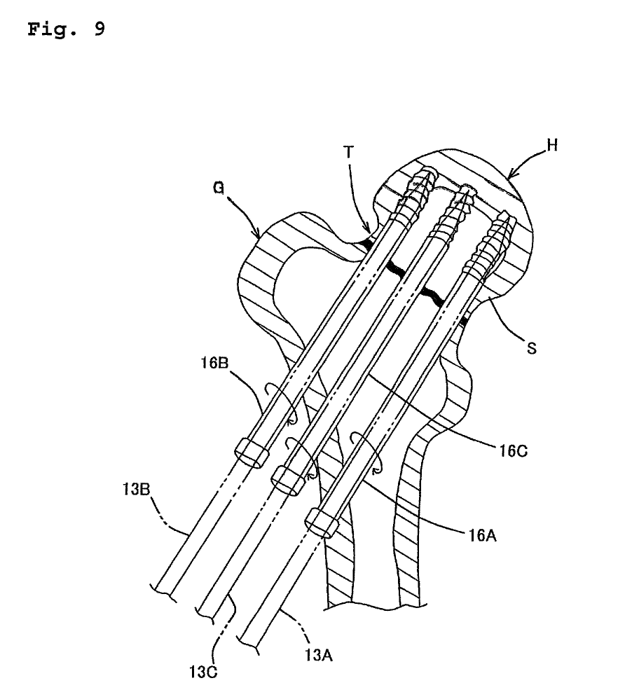

Secondarily, a first guide pin 13A of ø3.0 mm (a Kirschner's wire of ø3.0 mm) having a longer length is inserted in parallel with this wire 12 to reach the bone head “H”. In this procedure, the guide pin 13A should be inserted along the inner edge of the lateral cortical bone “S”, while checking the accurate insertion by the radioscopy. This is to ensure the reference position for a series of procedures for inserting a screw 16A along the direction of the guide pin 13A after the guide pin 13A having been inserted (see FIG. 9). When as a result of the series of procedures, the screw 16A would be successfully inserted so as to be brought into contact with the inner edge of the lateral cortical bone S in the femoral neck region “T”, the screw 16A can be supported by the lateral cortical bone S at a location most distant from the center of the femoral neck region T. Thereby, a rigid fixation can be obtained.

The insertion of the guide pin 13A may be accomplished by hammering the guide pin 13A with a hammer 14. Then, the Kirschner's wire 12 which is no more necessary can be pulled out.

Subsequently, as shown in FIG. 1, multi-slot guide 10 is mounted to the first guide pin 13A by inserting the first guide pin 13A into one of the pin inserting slots al of the multi-slot guide 10. Through this procedure, the multi-slot guide 10 has been installed on the first guide pin 13A.

In the condition where the first guide pin 13A is fittingly inserted in the pin inserting slot a1, an inserting point for a second guide pin 13B (the Krischner's wire of ø3.0 mm) is selected while rotating the multi-slot guide 10 in either direction around the guide pin 13A as the center of rotation. This selection is carried out to search for such a position for the second guide pin 13B that can touch the inner edge of the lateral cortical bone S in the femoral neck region T as a target, under checking by the radioscopy.

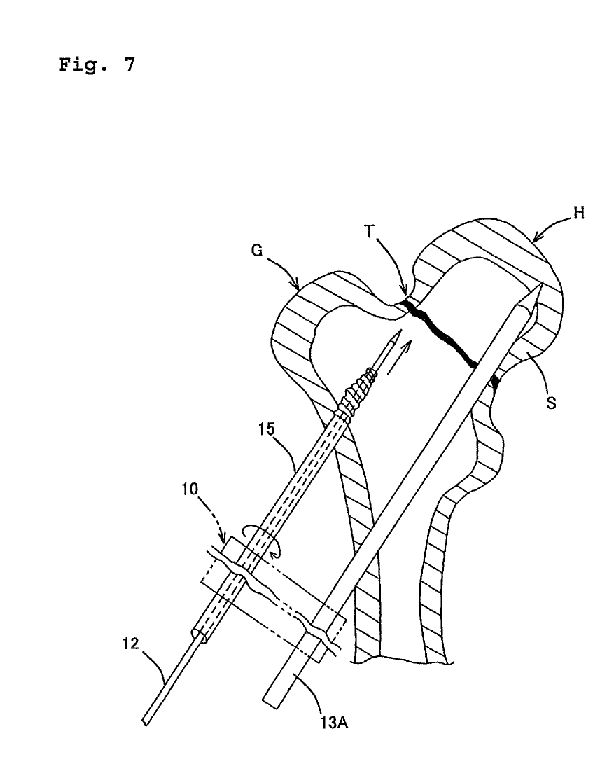

When, for example, the pin inserting slot d1 is selected as the inserting slot for the second guide pin, then a tubular cannulated drill 15 having an inner diameter of 1.5 mm and an outer diameter of 3.0 mm is introduced in the pin inserting slot d1 and the Kirschner's wire 12 is inserted into this cannulated drill 15, and then this wire 12 is operated to pierce the proximity of the base region of the greater trochanter “G”, as shown in FIG. 7. Upon this insertion, the Krischner's wire 12 should be inserted under the checking by the radioscopy so that the wire 12 may be brought into contact with the inner edge of the lateral cortical bone S in the femoral neck region T as much as possible. This is to ensure the reference position for a series of procedures for inserting a screw 16B along the direction of the guide pin 13B of ø3.0 mm after the guide pin 13B having been inserted. When as a result of the series of procedures, the screw 16B would be successfully inserted so as to be brought into contact with the inner edge of the lateral cortical bone S in the femoral neck region T, the screw 16B can be supported by the lateral cortical bone S at a location most distant from the center of the femoral neck region T. Thereby, the rigid fixation can be accomplished.

The Kirschner's wire 12 of ø1.5 mm is inserted toward the bone head, and the radioscopy is used to confirm that the Kirschner's wire 12 is in such a preferable position as being in contact with the inner edge of the lateral cortical bone S in the femoral neck region T. Thus, it is determined that this pin inserting slot d1 is a suitable pin inserting slot for the second guide pin.

It is to be appreciated that alternatively, the step for inserting the thinner Kirschner's wire 12 of ø1.5 mm so as to be brought into contact with the inner edge of the lateral cortical bone S in the femoral neck region T as much as possible may be omitted, and instead, the guide pin 13B with thicker diameter of ø3.0 mm may be directly inserted in the pin inserting slot d1. However, with this method applied, it is considered that the guide pin 13B may create a hole in the bone proximal to the base region of the greater trochanter G to advance toward the femoral neck region T. If it is confirmed through the radioscopy that the position of that guide pin 13B is out of the target, i.e., the guide pin 13B has not been inserted so as to be in contact with the inner edge of the lateral cortical bone S in the femoral neck region T, the guide pin 13B must be removed to make another trial of accurate insertion. This means that such a procedure for selecting the second pin inserting slot while rotating the multi-slot guide 10 in either direction around the first guide pin 13A as the center of rotation has to be repeated again. This may result in a big hole (bone-defected region) created in the proximity of the base region of the greater trochanter G. This would increase possibilities that this bone-defected region is subject to another fracture when the patient starts to walk with the load applied thereto after the operation.

In this viewpoint, the step of checking with the radioscopy the procedure for inserting the thinner Kirschner's wire 12 of ø1.5 mm so as to come in contact with the inner edge of the lateral cortical bone S in the femoral neck region T is required for no big holes (bone-defected regions) to be left in the bone cortex proximal to the greater trochanter G even in case of the retrial procedure being repeated.

Through the procedure as described above, when the pin inserting slot d1 has been determined to be suitable for that for the second guide pin, then the tubular cannulated drill 15 which has been installed in the multi-slot guide 10 is used to make a shallow drill hole of ø3.0 mm in the bone cortex proximal to the base region of the greater trochanter G with an aid of the Kirschner's wire 12 of ø1.5 mm, which has been inserted in the femoral neck region T and functions as the guide. Subsequently, the cannulated drill 15 and the Kirschner's wire 12 are pulled out. Then, the second guide pin 13B is inserted into the pin inserting slot d1 and hammered-in through this pierced drill hole of ø3.0 mm until the tip end thereof reaches a top portion of the bone head H.

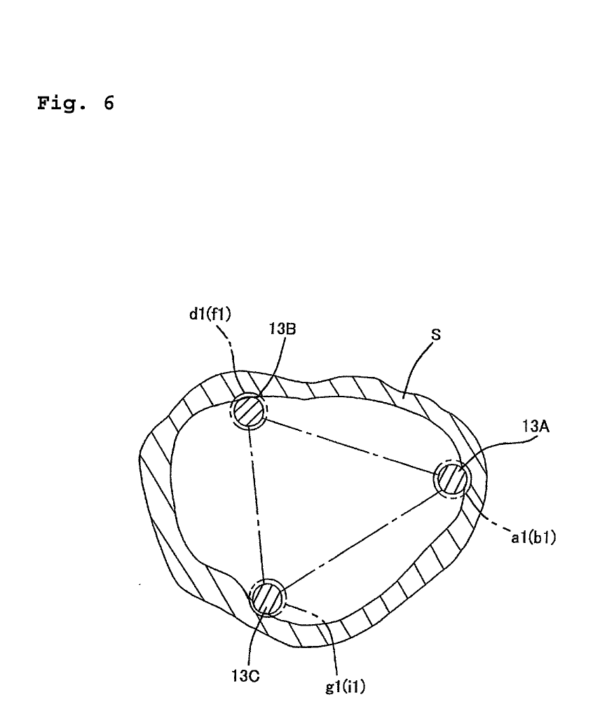

Then, though not shown, by performing the similar procedure to that for the second guide pin 13B, another pin inserting slot in the multi-slot guide 10 for the third guide pin (assuming the pin inserting slot g1 in this example) is selected and then the inserting procedure of the third guide pin 13C (the Kirschner's wire 12 of ø3.0 mm) into the upper region of the femur is carried out (see FIG. 6).

Subsequently, as shown in FIG. 9, the multi-slot guide 10 is pulled out of the guide pins 13A to 13C, and then with an aid of these guide pins 13A to 13C functioning as guiding axes, the three tubular screws 16 are screwed into the upper region of the femur while keeping the parallel relationships with one another and the triangle arrangement formed thereby.

As for the sequence for screwing, the screw 16A mounted on the first guide pin 13A is screwed in first, which is followed by the screw 16B mounted on the second guide pin 13B and then the screw 16C mounted on the third guide pin 13C. It is to be appreciated that the lengths of respective screws 16A to 16C should be determined by referring to the run-through length of the Kirschner's wire 12 of ø3.0 mm having the same length as that of the guide pin 13A, 13B and 13C (the Kirschner's wire of ø3.0 mm).

As a result of the procedures described above, the bone head H once separated from the upper region of the femur due to the fracture has been rigidly fixed to this upper region of the femur with three screws 16A to 16C.

As described above, since respective pin inserting slots a1 to j2 are formed penetrating through the multi-slot guide body so as to be disposed along respective extension lines connecting the imaginary center line L of the multi-slot guide 10 with respective vertexes of the imaginary triangles formed around this imaginary center line L such that a plurality of pin inserting slots is distributed along respective extension lines, a high level of fixation of the guide pins 13A to 13C and thus the screws 16A to 16C to the bone head H can be obtained irrespective of the size of the individual femur. Thereby, the earlier application of the load to the site of interest becomes possible and thus the period required until the patient starts walking can be reduced.

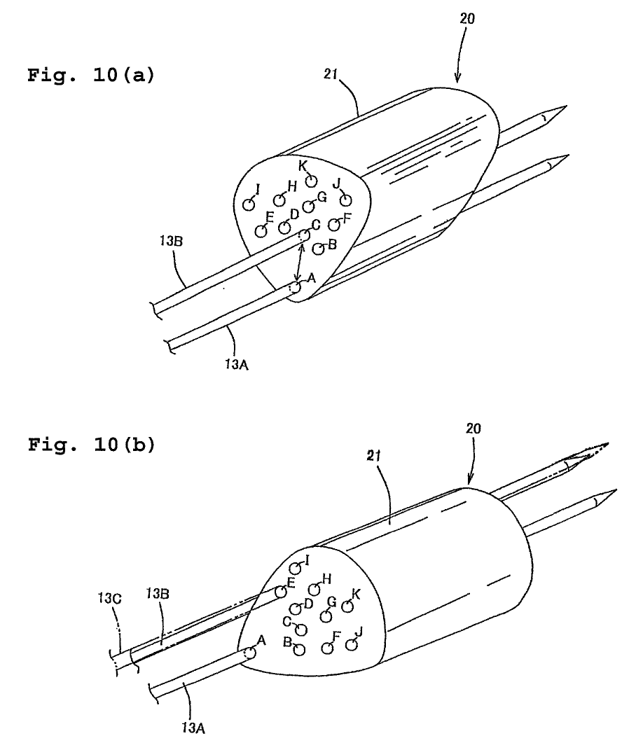

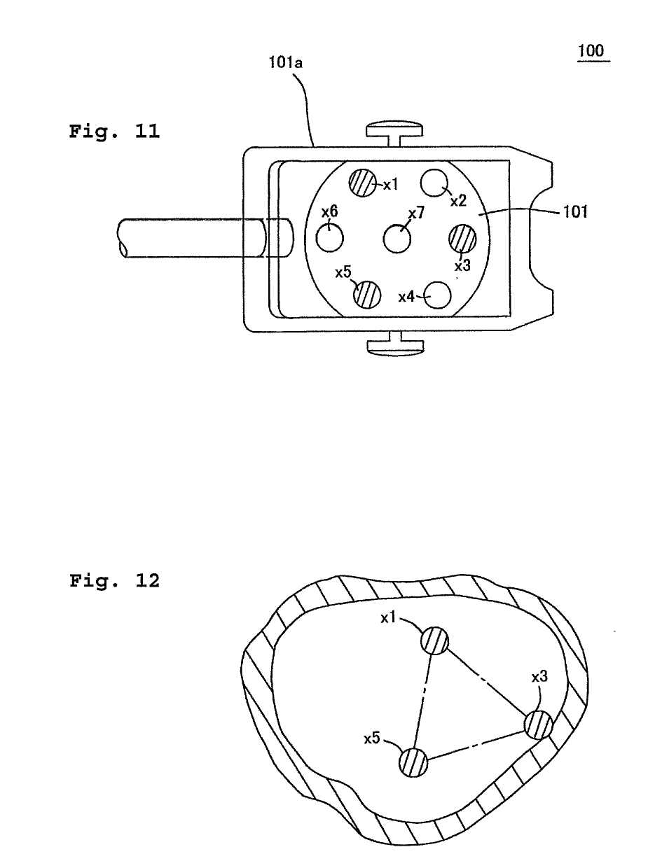

Now, a multi-slot guide for a femoral neck fracture bone-setting operation according to a second embodiment of the present invention will be described with reference to FIG. 10.

This multi-slot guide 20 according to the second embodiment is characterized in that the multi-slot guide 20 has a guide main body 21 in the form of short metal column and total of 11 pin inserting slots A to K, each having a diameter of ø3.0 mm, are formed in the guide main body 21 so as to penetrate through either end surface thereof in parallel relationships with one another.

The guide main body 21 is formed to have an approximately equilateral triangular section with respective edge lengths of 3.5 cm and a length of 6 cm along its axial line direction.

One pin inserting slot among these 11 pin inserting slots, which is located in the proximity to one vertex of the guide main body 21, is designated as a reference slot A, and the other tens lots are designated as differently distant slots B to K each having individual distance from this reference slot A different from one another respectively.

In more specific, the distances from the reference slot A are defined as followed: the distance between the pin inserting slots A and B as 12 mm, the distance between the pin inserting slots A and C as 14 mm, the distance between the pin inserting slots A and D as 16 mm, the distance between the pin inserting slots A and E as 18 mm, the distance between the pin inserting slots A and F as 20 mm, the distance between the pin inserting slots A and G as 22 mm, the distance between the pin inserting slots A and H as 24 mm, the distance between the pin inserting slots A and I as 26 mm, the distance between the pin inserting slots A and J as 28 mm, and the distance between the pin inserting slots A and K as 30 mm.

A femoral neck fracture bone-setting operation with the CCHS technique by using the multi-slot guide 20 of this second embodiment will now be described.

After a first guide pin 13A having been inserted along the lateral cortical bone S in the femoral neck region as shown in FIG. 5, the multi-slot guide 20 is mounted on the first guide pin 13A such that this first guide pin 13A may be inserted into the reference slot A.

Then, as shown in FIG. 10(a), with the first guide pin 13A inserted in the reference slot A, a differently distant slot for a second guide pin 13B that corresponds to another site of the lateral cortical bone in this femoral neck region T is selected (see FIG. 6). In this example, the differently distant slot C is selected.

A tubular cannulated drill having an inner diameter of 1.5 mm and an outer diameter of 3.0 mm is introduced into the differently distant slot C, and a Kirschner's wire 12 is inserted into the cannulated drill 15. This procedure is carried out while checking by the radioscopy such that the Kirschner's wire 12 may come in contact with the inner edge of the lateral cortical bone S over the range from the greater trochanter G to the femoral neck region T. At that time, in the same way as the aforementioned procedure, the multi-slot guide 20 may be rotated little by little in either direction until this Kirschner's wire 12 has been inserted into an appropriate position where it comes in contact with the inner edge of the lateral cortical bone in the femoral neck region T.

After having confirmed that the Kirschner's wire 12 has been successfully inserted to the targeted position by the radioscopy, the tubular cannulated drill 15, which has been installed in the differently distant slot C of the multi-slot guide 20, is operated to create a drill hole of ø3 mm in the bone cortex proximal to the base region of the greater trochanter G by expanding the drill hole toward a deeper direction by a small distance while keeping the Kirschner's wire 12 with the diameter of 1.5 mm inserted into the femoral neck region so as to function as a guide for the drilling. After that step, the cannulated drill 15 and the Kirschner's wire 12 are pulled out.

Subsequently, a second guide pin 13B is introduced in this differently distant slot C, and this second guide pin 13B is inserted from the base region of the greater trochanter G so as to be brought into contact with another site of the lateral cortical bone S in the femoral neck region T, and finally hammered into the bone head H by the hammer 14.

In next step, a procedure for selecting a differently distant slot for a third guide pin 13C is executed (see FIG. 6 again). In this example, it is assumed that the differently distant slot E has been selected.

The multi-slot guide 20 is removed once from the first and the second guide pins 13A, 13B, and then the first guide pin 13A is again fitted in the reference slot A. At that time, care must be taken to prevent the second guide pin 13B from being inserted in any one of the differently distant slots B to K.

In next step, with the first guide pin 13A inserted into the reference slot A, the multi-slot guide 20 is rotated in either direction around the guide pin 13A as the center of rotation. At that time, in the same way as the aforementioned procedure, the cannulated drill 15 is introduced in the differently distant slot E, and the Kirschner's wire 12 with a diameter of 1.5 mm is inserted into the drill 15. This procedure is repeated under the observation by the radioscopy until the Kirschner's wire 12 is finally brought into contact with the inner edge of the lateral cortical bone S, thus making a hole into which the guide pin 13C is to be inserted. After that, the cannulated drill 15 and the Kirschner's wire 12 are pulled out.

Although in this example, the differently distant slot E has been selected as the one into which the third guide pin 13C is inserted, the goal is a successful insertion of the guide pin 13C in the manner allowing the guide pin 13C to have a contact with the inner edge of the lateral cortical bone S. To accomplish this goal, the differently distant slot may be arbitrarily selected among those slots B to K. It is to be noted that preferably the slot should be selected so as to maximize the area of the triangle formed by the guide pins 13A to 13C.

Subsequently, via this differently distant slot E, the third guide pin 13C is hammered into the bone head H with the hammer 14 so that the guide pin can be brought into contact with the lateral cortical bone S in the femoral neck region T.

This allows the third guide pin 13C to be inserted smoothly and reliably in parallel with the other guide pins 13A and 13B. Further, with no regard to the size of the femur, this enables the guide pins 13A to 13C and thus the screws 16A to 16C directed to the bone head H to be rigidly fixed to the upper region of the femur. Thereby earlier application of the load to the affected site becomes possible.

Other configuration, operation and effect of the multi-slot guide of this second embodiment are the same as those of the first embodiment, and the description therefor should be omitted.

What is claimed is:

1. A multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins, which have been inserted from a base region of a femoral greater trochanter through the femoral neck region into the femoral head part, as guiding axes upon screwing-in,
said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them,
wherein said respective pin inserting slots are formed penetrating through said multi-slot guide so as to be disposed along extension lines connecting an imaginary center-axis line of said multi-slot guide for the femoral neck fracture bone-setting operation with respective vertexes of imaginary triangles formed around said center-axis line such that a plurality of pin inserting slot is formed along respective extension lines, said extension lines being arranged radially starting from said imaginary center-axis line at respective angle positions of 0 degree, 60 degree, 90 degree, 120 degree, 150 degree, 180 degree, 240 degree, 270 degree, 300 degree, and 330 degree.

2. A multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins which have been inserted from a base region of a femoral greater trochanter through the femoral neck region into the femoral head part as guiding axes upon screwing-in,
said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them,
wherein said respective pin inserting slots comprise one reference slot and a plurality of differently distant slots each having an individual distance from said reference slot different from one another respectively.

3. A multi-slot guide for a femoral neck fracture bone-setting operation, which is used in the cannulated cancellous hip screw technique for fixing a femoral head part once separated from an upper region of a femur due to a fracture in femoral neck region onto said upper region of the femur by screwing three tubular screws into the upper region of the femur, respectively, with utilizing corresponding three guide pins, which have been inserted from a base region of a femoral greater trochanter through the femoral neck region into the femoral head part, as guiding axes upon screwing-in,
said multi-slot guide having a plurality of pin inserting slots with respective slot axial directions being in parallel relationship with one another, through which said three guide pins are inserted into the femoral head part in such a manner that each of said three guide pins may contact with a cortical bone inner edge in the femoral neck region while keeping a triangular arrangement formed thereby and a parallel relationship among them,
wherein said multi-slot guide for a femoral neck fracture bone-setting operation is of cylindrical shape with approximately equilateral triangular section; and
said respective pin inserting slots comprise one reference slot formed in a proximity of one of vertexes of said approximately equilateral rectangular and a plurality of differently distant slots each being arranged radially around said reference slot with an individual distance from said reference slot different from one another respectively.

4. A multi-slot guide for a femoral neck fracture bone-setting operation in accordance with claim 3, in which distances of said differently distant slots defined from said reference slot increase by 2 mm in turn from shortest one.
